(19) United States
(12) Patent Application Publication
(10) Pub. No.: US 2007/0071762 A1
(43) Pub. Date: Mar. 29, 2007

(54) COMPREHENSIVE DIAGNOSTIC TESTING PROCEDURES FOR PERSONALIZED ANTICANCER CHEMOTHERAPY (PAC)

(75) Inventors: Paul O.P. Ts'o, Ellicott, MD (US); Stephen Lesko, Baltimore, MD (US)

Correspondence Address: Connolly Bove Lodge & Hutz LLP, Suite 800, 1990 M Street, N.W., Washington, DC, 20036, US

(73) Assignee: CCC Diagnostics, LLC, Ellicott City, MD (US)

(21) Appl. No.: 11/533,929

(22) Filed: Sep. 21, 2006

Related U.S. Application Data
(60) Provisional application No. 60/718,724, filed on Sep. 21, 2005.
(60) Provisional application No. 60/778,901, filed on Mar. 6, 2006.

(51) Int. Cl. G01N 33/574; A61K 39/395

(52) U.S. Cl. 424/155.1; 435/7.23

(57) ABSTRACT

The present invention provides methods of assessing and selecting treatment modalities for cancer.

### CROSS REFERENCE TO RELATED APPLICATIONS

This application claims priority to U.S. provisional patent application Ser. No. 60/718,724, filed on Sep. 21, 2005, and provisional patent application Ser. No. 60/778,901 filed on Mar. 6, 2006; the contents of both of which are specifically incorporated herein by reference.

### STATEMENT REGARDING FEDERALLY SPONSORED RESEARCH

Supported in part by NCI SBIR Grant CA081903 awarded to CCC Diagnostics, LLC. The government may have certain rights in the invention.

### TECHNICAL FIELD OF THE INVENTION

This invention is related to the area of cancer therapies. In particular, it relates to identifying the most efficacious therapy for cancer in individual patients.

### BACKGROUND OF THE INVENTION

Cancer is a highly individualized disease and the current favorable response rates for treatment with a single drug is low (˜20%). In order to increase the response rate, choosing the right drug for each patient is of utmost importance. It is well recognized that different patients respond in different ways to the same drug, most likely due to individual variability that results from genetic inheritance. Clinical observations of inherited differences in drug effects have given rise to the field of pharmacogenomics. A number of cases have been reported in which inter-individual differences in drug response are due to sequence variants in genes encoding drug-metabolizing enzymes, drug transporters or drug targets (for example, see Evans, W. E. Johnson, J. A. Annu. Rev. Genomics Hum. Genet. 2001; 2: 9-39 and McLeod, H. L. Evans, W. E. Annu. Rev. Pharmacol. Toxicol. 2001; 41: 101-121).

In addition to the heterogeneity of the host for the drug metabolism, there is the variation in tumors affecting the response of the tumor to a given drug, for example:
1) Most colon cancer patients respond to 5-FU chemotherapy if they have low levels of expression of thymidylate synthase, thymidine phosphorylase and dihydropyrimidine dehydrogenase. Patients have a very low response if the expression of one or more of the enzymes is at a high level;
2) Patients with beta-tubulin mutations do not respond well to paclitaxel-based chemotherapy while about 40% of patients with wild type beta-tubulin had complete or at least partial response. In addition, median survival also improved for patients carrying wild type beta-tubulin genes;
3) Either HER-2/neu gene amplification or strong overexpression (+3 by immunohistochemistry) of receptor protein can be used to identify a subset of patients that are more likely to respond to the combination of cytotoxic chemotherapy and trastuzumab. Single agent trastuzumab is active and well tolerated as a first-line treatment for women with metastatic breast cancer that demonstrates HER-2/neu overexpression or gene amplification; and
4) High nucleotide excision repair activity is closely correlated with cisplatin resistance in non-small lung cancer cells and an association between the level of ERCC-1 expression and repair of cisplatin-induced DNA damage in human ovarian cancer cells has been reported.

The above examples suggest that the level of expression of one or more specific biomarkers on cells of a tumor is related to response of the tumor to specific drugs. Since the more recent anticancer drugs are designed against specific cellular components (e.g., receptors, enzymes) in vital processes (e.g., repair, mitosis), it is likely biomarkers may be found which could indicate the response of the tumor cells when treated by the mechanistically related anticancer drug (see for example, Park, et al., Clinical Cancer Research 2004; 10:3885-3896 and Vande Woude, G. F. et al., Clinical Cancer Research 2004; 10:3897-3907).

It is generally recognized in the art that a need exists to match an individual's specific tumor to the most effective treatment modality, i.e., the need for Personalized Anticancer Chemotherapy (PAC). The present invention meets this and other needs.

### SUMMARY OF THE INVENTION

The invention establishes molecular diagnostic tests for personalized anticancer chemotherapy (PAC) via in vitro imaging technology. PAC involves the characterization of tumor cells obtained from an individual patient for drug response indicators/biomarkers (DRI). This approach is made favorable by the emergence of targeted chemotherapeutic drugs and the elucidation of tumor cell biomarker expression, which can be correlated with the resistance of the tumor to a mechanistically related drug. In vitro imaging of the tumor either as fresh cells or as archival cells preserved in paraffin blocks has been carried out with a computerized fluorescence microscopy system. Numerical measurements are normalized with fluorescent microspheres as reference.

In some embodiments, the present invention comprises obtaining one or more tumor cells from a patient and characterizing the tumor cells. Characterizing may include characterizing the tumor by antibodies, especially monoclonal antibodies. Typically such antibodies will be targeted toward tumor cellular components which are related to the mechanism of the action of the chemotherapeutic agents. The presence, absence or quantities of these components reflect the sensitivity or resistance of the tumor cells to the relevant drug(s) used in treatment. As used herein, a cellular component that is related to the mechanism of action of a particular chemotherapeutic agent may be considered a drug response indicator (DRI) for that chemotherapeutic agent.

In some embodiments, antibodies may be labeled, for example, each antibody may be labeled with a different fluorescent dye which has different excitation and nonoverlapping emission spectra for concurrent individual detection. The binding of these different fluorescently labeled antibodies may be measured, for example, may be measured quantitatively. In some embodiments, measurement may be made using a computerized fluorescence microscopy system by quantifying fluorescence intensity against a reference for standardization of the optical and recording system. The quantities of the various response indicators, corresponding specifically to various chemotherapeutic agents as measured by the fluorescence of the dye-labeled antibodies targeted to the drug response indicators in cells, may be compared from various relevant cancer cell lines exhibiting varying degrees of cytotoxic response to a given anticancer chemotherapeutic agent. The interpretation of the drug response indicator data from the patient's tumor is established by extrapolation to the drug response indicator data from various relevant cancer cell lines of varying cytotoxic sensitivities in responding to the pertinent drug treatment in culture. The predictive effectiveness of a chemotherapeutic agent is evaluated by noting the positive and/or negative influence of the drug response indicator pertaining to a given drug, i.e., the absence or presence, as well as the low quantity or the high quantity of certain drug response indicator(s) in tumor cells will cause the tumor not to respond (or not be sensitive) to a given drug treatment. This prediction can describe which government (FDA) approved chemotherapeutic agents most likely will not be effective against the tumor in an individual patient. Thus, in some embodiments, the selection of effective chemotherapeutic agent(s) for an individual patient is by excluding all the treatments with noneffective drugs as revealed by the drug response indicator of the tumor cells from individual cancer patients. The present invention confirms the direct correlation of the quantity of drug response indicators to the action of the drug by statistical analysis of cell culture data with drug treatment.

In some embodiments, a tumor cell sample may be obtained from the circulating cancer cells in the blood representing the metastatic cancer in the body. In some embodiments, a tumor cell sample may be obtained from the lymph nodes adjacent to the primary tumor as the cancer cells circulating in the lymphatic system, obtainable by means of biopsy such as bronchoscopic biopsy. In some embodiments, a tumor cell sample may be obtained from the primary tumor as the tumor tissue is obtained by biopsy or from surgical specimens. In some embodiments, such as when a sample is obtained from a primary tumor, a tumor tissue may be fixed in formalin and embedded in paraffin blocks and may be cut to thin sections, put on microscope slide for examination. In some embodiments, a section slide from a paraffin block of a tumor tissue may be deparaffinized by xylene and alcohol washing and may be processed through the antigen retrieval procedure, for example, with heating/hydroloysis/renaturing and may be then stained with appropriate fluorescently-labeled monoclonal antibodies against various cellular components for identification processes and for quantitative measurement of drug response indicators. In some embodiments, tissue on a processed slide may be examined, imaged, analyzed and recorded with a computerized fluorescence microscopic system. In some embodiments, 5 or more fluorescent antibodies may be measured, imaged, analyzed and recorded simultaneously from the same field of view on the section slide.

The present invention may be used to analyze cancers of any origin and any therapeutic modality. For example, cancer cell lines originating from breast, lung, colon and other cancer of epithelial cell origin that exhibit varying degrees of resistance (or sensitivity) to various government (FDA) approved cytotoxic agents. Cytotoxic agents may include, but are not limited to, carboplatin, cisplatin, oxaliplatin, docetaxel, paclitaxel, taxol, vinorelbine (vinca alkaloid), 5-fluouracil related drugs (such as xeloda), gemcitabine, and anthracycline. In some embodiments, an anticancer agent may comprise humanized monoclonal antibodies such as trastuzumab (herceptin), cetuximab (erbitux), and beracizumab (avastin).

In some embodiments, the drug response indicator comprises the following cellular components (antigen), and may be used to assess the effectiveness of the corresponding anticancer agents, which are approved by the FDA:

| Drug Response Indicators | Drugs |
|---|---|
| ERCC1 | Carboplatin |
| | Cisplatin |
| | Oxaloplatin |
| Estrogen Receptor | Tamoxifen |
| | Aromatase Inhibitors |
| β-tubulin III isoform | Docetaxel |
| | Paclitaxel |
| | Taxane |
| | Vinorelbine |
| Thymidylate Synthase | 5-FU related drugs |
| | Xeloda |
| Ribonucleotide Reductase | Germcitabine |
| Topoisomerase II | Anthracline |
| HER-2/neu Receptor, PTEN | Trastuzumab (Herceptin) |

In some embodiments, drug response indicators may comprise antigens targeted by the appropriately labeled monoclonal antibody therapeutic drugs, such as the appropriately fluorescently labeled trastuzumab (herceptin), cetuximab (erbitux), and beracizumab (avastin).

In one embodiment, the present invention provides a method of selecting a chemotherapeutic agent for treatment of cancer for an individual cancer patient comprising obtaining a tumor cell sample from the patient, determining a plurality of drug response indicators in the sample using antibodies; and selecting the chemotherapeutic agent. In some embodiments, the antibodies may be fluorescently labeled, for example, monoclonal antibodies. When antibodies are fluorescently labeled antibodies specific for each drug response indicator to be determined may be labeled with different fluorescent dyes having different excitation and nonoverlapping emission spectra permitting the simultaneous quantification of a plurality of drug response indicators. Typically, drug response indicators are cellular components related to the mechanism of action of chemotherapeutic agents and selection is based on the presence/absence or quantity of drug response indicator present in the cells of the sample. When the presence of one or more drug indicators is detected and it is desired to quantify the drug response indicators present, such quantifying may include comparison of the fluorescent intensity of the drug response indicator in the sample to one or more reference standards. Typically, method of the invention may comprise determining at least 5 drug response indicators. In some embodiments, determining comprises comparing the quantity of a plurality of drug response indicators in the sample, for example 5 or more, to a quantity of the same drug response indicators in cells of known response to chemotherapeutic agents that act through the drug response indicators. Any sample type may be used in the practice of the invention, for example, the sample may be obtained from circulating cancer cells in blood of the patient, obtained from lymph nodes adjacent to a primary tumor in the patient or obtained from a primary tumor in the patient. Methods of the invention may be used to select any suitable chemotherapeutic agent known in the art for example, carboplatin, cisplatin, oxaliplatin, docetaxel, paclitaxel, taxol, vinorelbine, vinca alkaloids, 5-fluouracil related drugs, xeloda, gemcitabine, anthracycline, humanized monoclonal antibodies, trastuzumab (herceptin), cetuximab (erbitux), and beracizumab (avastin). In some embodiments, at least one drug response indicator is ERCC1 and the chemotherapeutic agent is selected from the group consisting of Carboplatin, Cisplatin, and Oxaloplatin. In some embodiments, at least one drug response indicator is β-tubulin III isoform and the chemotherapeutic agent is selected from the group consisting of Docetaxel, Paclitaxel, Taxane, and Vinorelbine. In some embodiments, at least one drug response indicator is Thymidylate Synthase and the chemotherapeutic agent is selected from the group consisting of 5-FU related drugs, Leucovorin, Pemetrexel and Xeloda. In some embodiments, at least one drug response indicator is Topoisomerase II and the chemotherapeutic agent is selected from the group consisting of Anthracycline, Doxorubicin, and Epirubicin. In some embodiments, at least one drug response indicator is Topoisomerase I and the chemotherapeutic agent is Irinotecan. In some embodiments, at least one drug response indicator is ribonuclease reductase and the chemotherapeutic agent is Gemcitabine.

Three diagnostic tests have been established to implement this approach. These diagnostic tests are: (1) Drug Response Indicators Test (DRIT); (2) Herceptin-taxane Response Test (HER-Tax Test) for breast cancer patients who are HER-2/neu overexpression positive; (3) Circulating Cancer Cell Test (CCCT).

The DRIT and HER-Tax are tests for quantitative measurements of DRIs, while the CCCT measures the number of circulating cancer cells (CCC) and DRI biomarker expression in CCC. These three tests provide comprehensive information concerning the drug response of the tumors in individual patients, through which the most effective chemotherapeutic treatments can be selected.

For example, DRI expression levels in breast and lung cancer cells or in tumor tissue section slides can be measured utilizing monoclonal antibodies (MAB) labeled with fluorescent dyes to stain the specimens. An indexing system will be established to correlate the expression of DRI and the cytotoxic response (IC₅₀) of various cancer cell lines with varying resistance to the drug. This correlation of cytotoxic response is extended to DRI measurements of cancer cells embedded in paraffin blocks to establish a cell line standard which can serve as a reference for the expression of DRI in human tumor tissue sections cut from paraffin blocks, A reference range for each DRI measured in tumor sections can be constructed from a corresponding resistance/response probability to the drug based on the (IC₅₀) of the cancer cells in culture. A retrospective clinical study will be carried out to confirm this reference range for DRI index with recorded clinical outcomes. The innovation of this application is based on: (1) advances in in vitro imaging systems; (2) index system of cytotoxic responses correlated to the level of DRI; (3) the reference range for DRI index corresponding to tumor response as indicated by probabilities of tumor resistance. After consulting the DRI index of the patients, and the reference range of clinical response, the attending physician can make informed decisions about drug prescriptions for this patient.

### BRIEF DESCRIPTION OF THE DRAWINGS

FIG. 1 is a standard curve generated with an InSpeck Microscope Image Intensity Calibration Kit (six micron fluorescent microspheres) for use in comparing HER-2/neu quantitative data from various tumor cell preparations.

FIG. 2 is a digital image showing HER-2/neu fluorescence signal in a tissue section from a breast cancer patient. The section was cut from tumor tissue embedded in a paraffin block, processed for staining with Trastuzumab-Alexa 532, analyzed by fluorescence microscopy and imaged with a CCD camera.

FIG. 3 shows area of interest (AOI) regions of Trastuzumab-Alexa 532 stained cellular membranes selected for quantitation of HER-2/neu.

### DETAILED DESCRIPTION OF THE INVENTION

The term biological marker (biomarker) is defined as “a characteristic that is objectively measured and evaluated as an indicator of normal biological processes, pathogenic processes, or pharmacological responses to a therapeutic intervention”. For the purpose of this application, a drug response indicator in a cell is a biomarker that provides information of how the cell responds to the drug.

The present invention provides development of PAC based on: (1) Quantitative and simultaneous measurement of drug response indicators (DRI) in area of interest of each cell based on fluorescence intensity, normalized to a reference standard. The fluorescence indicates the quantity of the staining of the labeled MAB against the DRI. (2) Utilizing a battery of cancer cell lines with different resistance to various anticancer drugs, and statistically correlating the inhibition of in-vitro cell growth by each drug to DRI measurements pertaining to that drug. (3) A source of cancer cells and tumor tissue from individual cancer patients for measurement of DRI.

The development of PAC is driven by the low efficacy and substantial side effects of chemotherapeutic drugs, as well as high costs and time needed to develop new anticancer drugs. There is a great need to utilize the existing anticancer drugs more effectively for individual patients. The clinical application of PAC may provide one or more of the following benefits:
1) The prescription of a drug or set of drugs by the physician will be guided by an indexing system which provides reference to the probability of drug resistance of the prescribed drug for this patient indicated by the DRI expression of the patient.
2) The testing could be applied to several FDA-approved drugs (3-6) simultaneously, as well as several qualifying biomarkers for the same drug. In this fashion, ineffective drugs may be quickly eliminated from consideration as treatment and replaced by more effective drugs as an alternative.
3) The testing may be done in real time (2-3 days), rather inexpensive (less than one thousand dollars), large volume (up to a hundred tests per day for a dedicated laboratory) and can be carried out in a general hospital.
4) Imaging technology provides the basis for the quantitative measurement of the DRI value of the tumor, including evaluation of the tumor heterogeneity. This measurement of heterogeneity may indicate the duration of response of this patient to this drug.

From the careful survey of the literature, a list of FDA approved, widely utilized drugs for breast, lung and colon cancer and their respectively, mechanistically related DRI are listed in the following table.

| Anticancer Drug | DRI | Type of Cancer | Comments |
|---|---|---|---|
| Herceptin | Her-2/neu Receptor | Breast | FDA-required |
| | | | confirmatory. |
| | PTEN | Breast | Another factor defining the |
| | | | response to Herceptin. |
| Tamoxifen | Estrogen Receptor | Breast | Well accepted and |
| | | | established confirmatory. |
| Aromatase Inhibitor | Estrogen Receptor | Breast | |
| Taxane Family | Class III β-tubulin | Breast | Class I II & IV β-tubulin |
| | | | isoforms are reported to be |
| | | | related. |
| Docetaxel | | Colon | |
| Pacitaxel | | NSC-Lung | |
| Taxol | | | |
| Vinca alkaloids | | | |
| Gemcitabine | Ribonucleotide | Lung | Often used in combination |
| | Reductase (RR) | | with platinum, taxane. |
| Platinum Families | ERCCI | | Major drug for lung |
| | | | cancer. |
| | | | Often used in combination |
| | | | with 5-FU, taxane, etc. |
| Oxaliplatin | ERCCI | Colon | Oxaliplatin is for colon and |
| | | | cisplatin and carboplatin |
| | | | for lung. |
| Cisplatin and | | NSC-Lung | XPP or ERCCII have also |
| Carboplatin | | | been used as DRI. |
| 5-FU Family | Thymidylate | Breast | |
| | synthase (TS) | | |
| 5-FU | Thymidylate | Colon | Often used in combination |
| | Phosphorylase (TP) | | with oxaliplatin irinotecin. |
| FUDR | Dihydropyrimidine | | |
| Capecitabine | Dehydrogenase | | |
| | (DPD) | | |
| Anthracycline | Topoisomerase II | Breast | Often used drug- |
| | | | exploratory. |
| Doxorubicin(and | | | |
| adriarmycin) | | | |

Additional drug response indicators are provided in the following table that provides Drug Response Indicators (DRI) to the Targeted Anticancer Chemotherapy for 6 Cancers The listed chemotherapy came from the National Comprehensive Cancer Network (NCCN) Clinical Practice Guidelines in Oncology, 2006. The DRIs related to the chemotherapy came from the literature

| Drug Response | | | | | | |
|---|---|---|---|---|---|---|
| Indicators | Breast | Lung | Colon | Gastric | Pancreas | Esophagus |
| HER-2/neu Receptor | Trastuzumab/Herceptin | | | | | |
| Estrogen Receptor | Tamoxifen | | | | | |
| | Armotase Inhibitor | | | | | |
| B-tubulin III isoform | Taxane-based | Taxane-based | | Taxane-based | | Taxane- |
| | Docetaxel | Docetaxel* | | | | based |
| | Paclitaxel | Paclitaxel* | | | | |
| | Vinorelbine | Vinorelbine | | | | |
| ERCC-1 | Cisplatin | Cisplatin* | Oxaliplatin | Cisplatin | Oxalipatin | Cisplatin |
| | Carboplatin | Carboplatin* | | Oxalipatin | Cisplatin | Oxalipatin |
| Thymidylate Synthase | Xeloda | Premetrexed(antifolate) | 5-FU | 5-FU | 5-FU | 5-FU |
| Thymidine | 5-FU | | Xeloda | Xeloda | Xeloda | Xeloda |
| Phosphorylase | | | Leucovorin | Pemetrexel | | |
| Ribonucleotide | Gemcitabine | Gemcitabine* | | | Gemcitabine | |
| reductase | | | | | based | |
| Topoisomerase II | Anthracycline | Doxorubicin³ | | Epirubicin | Epirubicin | |
| | Doxorubicin | | | Anthracycline | | |
| Topoisomerase I | | Irinotecan* | Irinotecan | Irinotecan | | Irinotecan |

Notes:
1) DRI can be determined from fresh cancer cells in biopsy after fixation in paraformaldehyde and from sections of tumor tissue fixed in formalin and embedded in paraffin blocks.
2) The correlative clinical studies for the DRI Tests will be conducted with Stage IV or metastatic patients. A more defined clinical response can be made from the size or number of their tumors from this cohort of patients. The results, however, are applicable for chemotherapy at all stages, including adjuvant stage.
³For lung cancer, these drugs are used for both small cell lung cancer (indicated by an asterisk) as well as by non-small cell lung cancer, except doxorubicin is used for the small cell lung cancer.
4) Bevacizumab (Avastin) is used in combination on breast, colon, lung cancers. A separate DRI Test can be established for the evaluation of the binding of Avastin to tumor cells.

As mentioned above, a battery of cancer cell lines with different resistance to various anticancer drugs is required to set up the index system. Tumors derived immortal cell lines generally display robust proliferation and fill a need for functional cancer cell model systems. Cancer cell lines have been utilized for prediction of responses to anticancer drugs with some degree of success. In one study, 39 human cancer cell lines were analyzed in respect to their sensitivities to 55 cytotoxic cancer drugs. It was concluded that the integrated database of gene expression and chemosensitivity profile might be useful to develop systems for the prediction of drug efficacy. In another study, the Phase II trial results of 31 cytotoxic drugs were correlated with the screening of The National Cancer Institute Human Tumor Cell Line Panel. It was concluded that the in vitro cell line model was predictive for non-small cell lung cancer, breast cancer and ovarian cancer in different approaches. While neither study specified a system for clinical application, they indicated that the cytotoxic sensitivities of human cancer lines can be useful for correlation or prediction of clinical trial results

The effective application of targeted anticancer therapy is to couple it with molecular diagnostic technology to form Personalized Anticancer Chemotherapy (PAC). PAC is currently the most promising development for cancer treatment. HER-2/neu expression coupled with trastuzumab treatment for metastatic breast cancer (MBC) patients is the first FDA approved Rx-Dx coupling and serves as a model system for PAC.

The present invention involves the utilization of in vitro imaging technology and molecular diagnostics in the characterization of tumor cells obtained from an individual patient for quantifying drug response indicators/biomarkers (DRI) and using this information to select an appropriate therapeutic modality for the patient. The molecular diagnostic procedure is comprised of the following three steps,
(1) Acquiring cancer cell sample(s) from individual cancer patients; these cancer cells can be fresh cancer cells obtained from frozen tumor sections, biopsy material, circulating cancer cells in the blood, or archival cells obtained from serial sections cut from formalin fixed tumor tissue embedded in paraffin blocks.
(2) Quantitatively assessing the biomarkers (drug response indicators, DRI) expressed in the tumor cells since the level of expression of these biomarkers is related to the resistance of the tumor cells to a given targeted therapy. Assessing may include the application of appropriately labeled fluorescent monoclonal antibody (Mab) targeted toward cellular biomarkers, which are responsible for the resistance of the tumor cells to a targeted therapy. The quantitation may be accomplished with a computerized fluorescence microscopy system, the proper software, and a reference standard.
(3) Selecting an appropriate therapeutic modality based upon a correlation of drug response indicator expression in tumor cells to the cytotoxic response of tumor cells to a mechanistically related drug. A battery of relevant cancer cell lines with different resistance to various anticancer drugs can be utilized. The effect of these drugs on the proliferation of the cancer lines in culture is determined together with the level of expression of the corresponding DRI in these cell lines. This data allows for the interpretation of DRI expression levels (by fluorescence) of tumor cells as correlated to tumor resistance to a drug for targeted therapy. The in vitro drug cytotoxicity-DRI relationship forms the experimental basis for extending the correlation of DRI expression to a given drug with the clinical response of a given patient to treatment with that drug. This correlation is extended to DRI measurements of cancer cells embedded in paraffin blocks, which can serve as reference for the expression of DRI in human tumor tissue sections cut from paraffin blocks. A reference range of each DRI measured in tumor sections can be constructed which shows a corresponding resistance/response probability to the drug based on the (IC₅₀) of the cancer cells in culture.

The prediction of the efficacy of targeted therapy as applied to a tumor(s) of a patient is based on the positive and/or negative influence of DRI in the tumor cells, i.e., that the absence/presence or low/high quantity of DRI in tumor cells will cause the tumor not to respond to a given drug treatment. Thus, the selection of effective targeted therapy for a patient is accomplished by excluding all treatments with noneffective drugs as revealed by the DRI measurements of tumor cells from the cancer patient.

The duration of response to a given drug by the tumor of individual patients can also be evaluated by the percentage of tumor cells which are resistant to the drug versus the percentage of tumor cells which are not resistant to the drug (heterogeneity of the tumor). When the nonresistant portion of the tumor is attacked and killed off by the drug, then the remaining resistant cells become the dominant portion and the whole tumor becomes resistant to the drug. This reasoning suggests the heterogeneity measurement of the tumor would be a marker of resistance, and further indicate that another effective drug has to be used as a combination and/or a follow up treatment in order to prolong the survival of the patient.

In the clinical application, response rate (RR) and/or time to progression (TTP) of patients may be statistically correlated with the DRI measurement of their tumors and a reference range of probability of resistance (nonresponsiveness) for each individual patient at different DRI indexes will be constructed. After consulting the DRI index of the individual patient, and the reference range of clinical response, the attending physician can make informed decisions about drug prescription for this patient.

In order to implement the approach of PAC, three diagnostic tests have been established for the service of the cancer patients. These diagnostic tests are: (1) Drug Response Indicators Test (DRIT); (2) Herceptin-taxane response test (HER-TAX Test); and (3) The Circulating Cancer Cell Test (CCCT). These tests will be further described below.

Cancerous cells have two distinct characteristics:
1) replication not under control of the host becomes a tumor, and
2) the tumor enlarges, spreads and obstructs the vital functions of the body leading to mortality.

When the cancer/tumor can not be removed or destroyed by physical force (surgery or irradiation), then a systemic anticancer chemotherapy is needed.

The key to the success of anticancer chemotherapy is the ability to destroy the cancer cells without harming the normal cells, which are the absolute majority of cells in the host. Since uncontrollable replication is characteristic of cancer cells as well as being obstructive to normal functions, most of the cytotoxic drugs directed against cancer are designed to attack the cellular entities and processes involved in the replication process.

The modern anticancer cytotoxic drugs are targeted therapies, aiming at cell components involving a vital process, such as DNA replicating enzymes, nucleotide (building blocks of nucleic acids) enzymes, DNA repair enzymes, receptors for transmitting replication signals, etc. From these key protein moieties, antibodies, especially monoclonal antibodies (Mab), can be readily generated which can have a high affinity constant in the range of 10¹¹⁻¹³ Mol⁻¹. Thus, these Mab can selectively and tightly bind to these target proteins. Also, when these Mab are chemically linked to fluorescent dyes, the quantities and locations of the fluorescent Mab-target complexes can be detected, imaged, and recorded. When different Mab are each labeled with different fluorescent dyes possessing different and nonoverlapping excitation/emission spectral positions, these different Mab-targets can be imaged in the region of interest (ROI) simultaneously but with separate detection.

One effective instrument for measuring fluorescent complexes is a computerized, fluorescence microscopy system (FMS). The areas inside the cell containing these complexes can be defined, and the measurement of the fluorescence can be expressed in total fluorescence of a defined spatial region or as the average fluorescence per pixel, or even the maximum intensity per pixel in this region. This capability will be shown in the Example 1.

In addition to having a numerically quantifiable measurement, the measurement by the FMS can be standardized through the use of fluorescent microspheres. After the calibration of FMS at different wavelengths via the fluorescent microsphere, the FMS measurement can be compared both in a temporal sense, and from different FMS. This operation will be shown in the Example 2.

Tumor tissue from individual patients is obtained from three sources generally: (1) the biopsy material from probing the tumor or the lymph nodes adjacent to the tumor, (2) the surgical material obtained from the operation removing the tumor, (3) circulating cancer cells in the blood which represent the metastatic tumor. We have examined the tumor cells from all these sources, and each source presents its unique requirement. For characterizing tumor cells from biopsy, the most important requirement is to identify the tumor cells from normal cells. When the tumor cells are epithelial in origin, and the surrounding cells are not (blood cells or lymphatic cells), then the identification is relatively straightforward via the characteristic cytokeratin skeleton of epithelial cells. Mab specifically against the proteins in cytoskeleton are available. An enrichment process is used for characterizing circulating tumor cells from the blood through which most of the normal blood cells are excluded, with the cancerous epithelial cells left behind for characterization. Most commonly, the tumor tissue is collected from the surgical specimen; these tissues are fixed in formalin and embedded in paraffin blocks and stored. Section slides can then be obtained from these blocks for viewing after a process of de-paraffinization, washing, and antigen retrieval. Example 3 shows these preparations. Each antigen, or even protein target, may require a separate activating/retrieval procedure.

In Example 4 we shall describe the experimental procedure for the quantitative measurement and recording of the fluorescent Mab-antigen complex in the viewing area (ROI) of the section slide by the FMS. The procedure requires the subtraction by the computerized system of the background autofluorescence. This background is obtained from FMS in viewing a similar area by the serial sectioning of the paraffin block without the staining by the fluorescent Mab. Otherwise, the optical measurement process remains the same and the information concerning the background is stored in the computer to be used subsequently for the subtraction as the background.

Example 5 describes staining and numerical measurement of drug response indicators in tissue sections of formalin-fixed tumor embedded in paraffin blocks.

Example 6 describes the assessment of heterogeneity within different areas of a tumor section. The duration of response to a given drug by the tumor of individual patients can also be evaluated by the percentage of tumor cells which are resistant to the drug versus the percentage of tumor cells which are not resistant to the drug (heterogeneity of the tumor). The heterogeneity measurement of the tumor would constitute a marker of resistance, and further indicate that another effective drug has to be used as a follow up treatment in order to prolong the survival of the patient.

The construction of a DRI index to correlate the cytotoxic action of chemotherapeutic drugs with the expression of mechanistically related DRI is described in Example 7. This method establishes statistically significant correlations between responsiveness of cultured human tumor cells to chemotherapeutic agents and the expression of DRI that are mechanistically related to the mode of action of the drug. Statistical analysis will be performed as described in Example 9 to correlate these two values and to establish a DRI expression level that may be used as an index to indicate clinical response.

The extension of the above in vitro indexing system to the construction of a DRI Index system based on paraffin embedded cultured cell standard is described in Example 8. This technique establishes a control standard that reflects the influences of tissue fixation and processing.

Three molecular diagnostic tests that may provide information for management of the disease for individual cancer patients are detailed in Example 10. In order to implement the approach of PAC, these diagnostic tests have been established for the service of the cancer patients. The diagnostic tests are: (1) Drug Response Indicators Test (DRIT); (2) Herceptin-taxane response test (HER-TAX Test); and (3) The Circulating Cancer Cell Test (CCCT). Each is described in detail in the example.

Example 11 details the steps necessary for the establishment of PAC in Breast cancer patients and provides pertinent data to support this strategy.

The clinical correlative study design necessary to confirm the PAC system is described in Example 12. A retrospective study will be performed first to correlate known clinical results with the measurement of DRI from tumor cells of those same patients. Subsequently, a prospective study of DRI data will be correlated with the results of patients under treatment. The data from this trial will serve as IDE and will lead to PMA from FDA.

A situation where the above described strategy works particularly well is the use of humanized monoclonal antibody therapeutic agents such as trastuzumab (herceptin) for breast cancer and cetuximab (erbitux) for colon cancer. In this approach, we have used the monoclonal drug itself as a probe for the target-receptor after attachment of a fluorescent dye onto the therapeutic Mab. Obviously, if the therapeutic Mab cannot attach to the tumor cells, the tumor cells would not respond to the therapeutic Mab. It should be noted that the converse may not be true, that is, the attachment of Mab to the tumor cells may not bring cytotoxic response. Thus, the essential information derived from this approach of quantitative measurement of drug response indicators is the determination of which cell will not show cytotoxic response to the drug because of the absence of the drug response indicator. However, the favorable measurement of the drug response indicators may not provide assured information that these tumor cells will show cytotoxic response. In other words, the negative drug response indicators would predict that the tumor cells will not exhibit cytotoxic response, but a positive drug response indicator would not assure that the tumor cells will exhibit cytotoxic response because the influence of other important factors. Thus, we can have an “ineffective drug indicator” to exclude the use of ineffective (but still exhibiting side effects) drugs for a tumor in an individual patient. The knowledge of ineffective drug indicators is very useful and reliable, as this conclusion is supported statistically under a defined set of conditions in cell cultures.

In general, the above description outlines the components involved in “Personalized Anticancer Therapy” (PAC). First, a targeted therapeutic drug is developed and approved by FDA. Second, an essential cellular target has been identified in laboratory and in clinical studies. Third, an antibody (a particular monoclonal antibody) directed specifically against this protein target has been generated and labeled with appropriate fluorescent dyes. Fourth, a computerized fluorescence microscopy system is assembled to measure, image, and record the quantities and location of these fluorescent Mab-target complexes in the tumor cells. Fifth, a source of representative tumor cells can be obtained either through biopsy procedure, surgical specimen, or circulating tumor cells in the blood. Sixth, selecting an appropriate chemotherapeutic using a correlation study done to demonstrate in a well-defined in vitro situation that the quantity of a drug response marker is statistically correlated with the cytotoxic response of tumor cells.

The connection of these six components forms the basis of “Personalized Anticancer Therapy”, with informed recommendation to exclude the use of those ineffective drugs for a given cancer patient. This strategy has been demonstrated in clinical correlative studies the medical contribution of the information concerning “ineffective drug indicators.” An initial stage of this demonstration has been given in the FDA approved procedure for the use of trastuzumab, and will be described in a separate section.

In putting the PAC systems together, we have provided the procedures in the application of FMS in measuring the biomarkers quantitatively from the tumor cells obtained from individual patients. Specific procedures for the tumor cells obtained from different sources will be described in the examples.

### EXAMPLE 1

Measurement of Fluorescent Complexes in Fixed Cells on Microscope Slides

Our system is a computer assisted Leica DMRXA fluorescence microscope equipped with a CCD camera, an eight-filter cube turret and Image-Pro Plus software for image acquisition and image processing. Currently, we can measure the fluorescence of five spectral regions from the same cell; two are used for cell identification and three are used to measure biomarkers. In order to stain cells with multiple mouse monoclonal antibodies it is necessary to directly label each antibody. This is accomplished with fluorescein isothiocyanate and succinimidyl ester derivatives of Alexa dyes from Molecular Probes.

In this example, the quantification of HER-2/neu in a breast cancer cell line is demonstrated. The monoclonal antibody, Trastuzumab, was labeled with Alexa 532 and anti-pancytokeratin was labeled with FITC. A breast cancer cell line (SKBR-3) was incubated with the above antibodies, then washed and mounted for counter-staining with DAPI in an antifade medium. Digital images were acquired at the appropriate exposure time using filter cubes that allow for discrimination of DAPI, FITC and Alex 532 signals in the same cells. The spatial area of each cell was outlined using the cytokeratin fluorescence. These outlines are saved, recalled and overlaid on the Alexa 532 image, i.e., the HER-2/neu signal. The following table presents quantitative data for HER-2/neu expression in four cells. Column 1 gives the object number, Column 2 presents the cellular area as number of pixels, Column 3 gives the average fluorescence intensity per pixel in each cell and Column 4 give the integrated fluorescence intensity for each cell. The data indicate that HER-2/neu expression in cells can be variable.

| | | Fluorescence | Total |
|---|---|---|---|
| Cell no. | Area in pixels | intensity per pixel | fluorescence |
| 1 | 4444 | 362.2061 | 1609644 |
| 2 | 3783 | 314.2744 | 1188900 |
| 3 | 3130 | 395.1147 | 1236709 |
| 4 | 3126 | 233.0742 | 728590 |

### EXAMPLE 2

Calibration of Microscopy System.

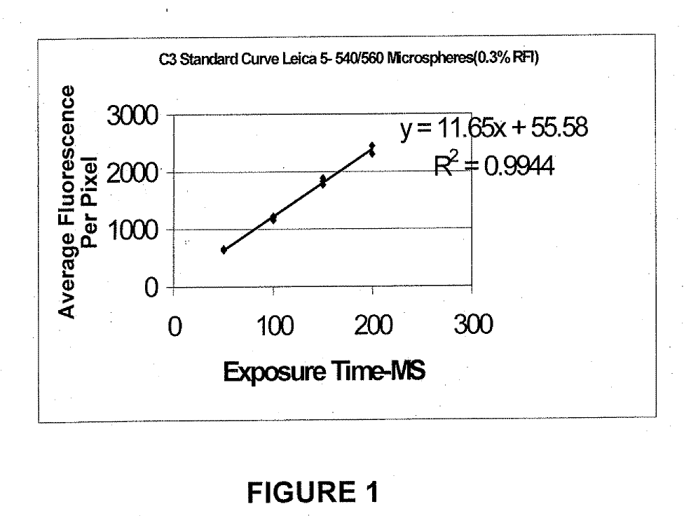

To conduct quantitative immunofluorescence studies one must be able to acquire digital images and compare fluorescence measurements obtained over various time periods and on different microscopes. This was accomplished by calibrating our Leica microscopy systems with a readily available fluorescence standard obtained from Molecular Probes. It is composed of six micron-diameter fluorescence microspheres (Inspeck Microscopy Image Intensity Calibration Kit). A suspension of microspheres is placed on a microscope slide, air dried and mounted in anti-fade medium under a coverslip. Images are acquired at various exposure times being sure not to exceed times that result in a saturation level, i.e., 4096 fluorescence units per pixel for 12-bit images. The images are processed with Image-Pro Plus software to obtain the average fluorescence intensity per pixel of about 4 or 5 microspheres at each exposure time. Standard curves are obtained for each filter cube by plotting average fluorescence intensity per pixel against exposure time in milliseconds. An example of such a linear standard curve is presented in FIG. 1. The slope and intercept are used to calculate the exposure time required to yield an average fluorescence of 2000 units with the reference standard; for the plot shown in FIG. 1 a value of 176 milliseconds was obtained. Thus, by using the same fluorescence standard, each microscope/filter cube can be calibrated to give the same fluorescence intensity by selecting the appropriate exposure time.

### EXAMPLE 3

Preparation and Staining of Tumor Cell Preparations for Identification and Characterization.

Lung cancer cells can be obtained by bronchoscopic biopsy. Biopsy material may be put into neutral saline after removal from patient. The cells may be washed in PBS and brought up to a specified volume for counting. An appropriate number of cells may be deposited on a microscope slide within a PapPen-outlined area. After drying, the cells may be fixed in 2% paraformaldehyde, incubated with anti-pancytokeratin-FITC and counterstained with DAPI to identify epithelial cells. Images may be acquired and the number of epithelial cells with intact nuclei counted. Tumor cells can be distinguished from normal epithelial cells by determining the epithelial cell/wbc nuclear DNA ratio of the cells on the slide as a measure of aneuploidy and by quantifying the expression of alpha fetoprotein receptor in the epithelial cells by staining with a specific monoclonal antibody. Quantiation utilizing fluorescently labeled antibodies may be performed as described in Example 1.

Tissue sections obtained during surgery or by biopsy may be fixed in formalin and embedded in paraffin blocks. Serial four micron sections may be cut from these paraffin blocks and mounted on microscope slides. The section may be deparaffinized as follows: twice in xylene, 5 minutes each; twice in 95% ethanol, 3 minutes each; twice in 70% ethanol, 3 minutes each; then in tap water for at least 10 minutes. For antigen retrieval, slides may be heated in 10 mM EDTA, pH 8, or 10 mM citrate, pH 6, for 30 minutes at 95 degrees followed by 20 minutes in the same solution placed at room temperature. The slides may be washed in PBS and stained with anti-pancytokeratin-FITC and other fluorescently labeled antibodies of choice.

Circulating cancer cells (CCC) in the blood can be isolated and identified through the following protocol: Enrich the cancer cells from 15-20 ml of blood using double-gradient centrifugation followed by immunomagnetic beads to remove most of the blood cells (negative selection). Deposit the cells on a microscope slide within a PapPen-outline area and incubate with an antibody cocktail (FITC-labeled antibodies with reactivity against nine cytokeratin peptides and a tumor-associated glycoprotein expressed on human carcinomas). Counterstain by mounting with DAPI-containing anti-fade medium. Scan slides with a fluorescence microscope and enumerate FITC positive cells with intact nuclei.

In the standard CCC test, over 100,000 white blood cells (WBC) are recovered with the CCC. The WBC interfere with the staining of cancer cells by the dye-MAB complex in some cases (but not with HER-2/neu or cytokeratin). Therefore, a Superenrichment procedure was developed which results in the recovery of less than 3000 WBC with the cancer cells. In these preparations, the staining of the cells is not influenced by the small number of WBC. This procedure is detailed below:

CCC SuperEnrichment Protocol:
Dilute 15-20 ml anticoagulated peripheral blood (6-hr, spiked with 100 MCF-7 breast cancer cells) with PBS up to total 30 ml;
Carefully layer diluted cell suspension over 15 mL 1.083 gradient in 50-ml conical tube and centrifuge at 2000 rpm 30 min at 20 CC in a swinging-bucket rotor (without brake);
Pipette off supernatant completely and collect 6-8 ml upper portion of 1.0830 interface;
Wash twice with Hanks' Solution by centrifuging at 1200 rpm for 10 min;
Carefully remove supernatant completely;
Add 1× dilution buffer to final volume of 40 ml and mix well;
Add 5 ml of MACS CellPerm Solution, mix well and incubate for EXACTLY 5 min at RT, following by adding 5 ml of MACS CellFix Solution, mix well and incubate for 30 min;
Centrifuge cell suspension at 1200 rpm for 10 min;
Resuspend cell pellet in 1×MACS CellStain Solution in a final volume of 600 μl;
Add 200 μl of FcR Blocking Reagent and mix well;
Add 200 μl of MACS CK Microbeads and incubate for 45 min at 20-25° C.;
Add 100 μl of anti-Cytokeratin-FITC and incubate for additional 10 min;
Add 4 ml of 1× MACS Cell Stain Solution and centrifuge cell suspension at 1200 rpm;
Place a positive selection column in the magnetic field of the MACS separator;
Apply resuspended cells to the column, allow white blood cells (WBC) to pass through the column and wash with 3×500 μl degassed 1× Dilution Buffer;
Remove column from separator, place column on 15 ml tube;
Pipette 1 ml of degassed 1× Dilution Buffer on top of column and elute retained circulating cancer cells using the plunger applied with column;
Spin down the cell pellet and direct-deposit on slide air dry at RT for 8-24 hr; and
Add DAPI in mounting median and subject sample to computer assisted microscopy analysis.

### EXAMPLE 4

Quantifying Biomarker Expression in Tumor Tissue Sections Cut from Paraffin Blocks.

The fluorescence intensity measured across the tissue section reflects the amount of fluorescently labeled primary antibody bound to the antigen, which in turn represents the amount of targeted protein (biomarker) in the cells. However, autofluorescence inherent in formalin-fixed tissue needs to be accounted for in order to generate reproducible quantitative data. Autofluorescence is measured by processing a control slide, a serial section of the same tumor, in the protocol but without the fluorescently labeled primary antibody.

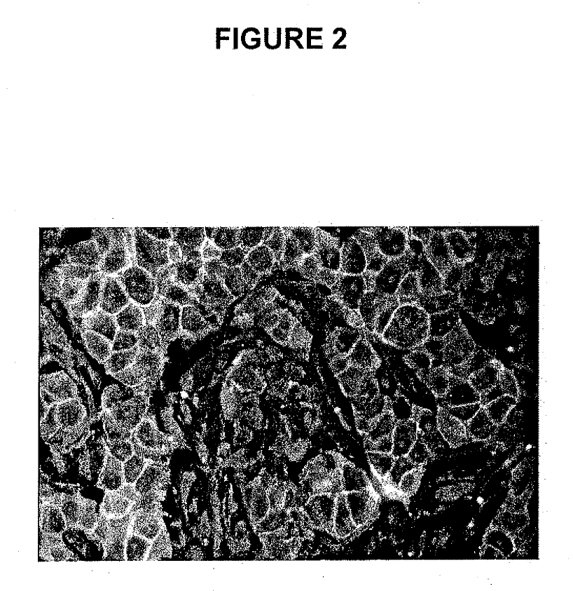

Digital images of three to four different areas from each tumor are acquired C1 (DAPI), C2 (FITC), C3 (Alexa 532), C4 (Alexa 594) and C5 (Alexa 647) filter cubes using exposure times that would yield 2000 fluorescence units with our reference standard (see Example 2). Control and experimental slides are treated exactly alike. In addition, an image is obtained with each filter of a background area on the slide which does not contain tissue. The images are processed with Image-Pro Plus software. The average fluorescence per pixel for each background image is obtained from a histogram and that value is subtracted from the appropriate experimental image. Three to four areas of interest (AOI), each containing about 10 cells, on each image are selected for quantification of the biomarkers. A representative image of a HER-2/neu-stained breast tumor section is presented in FIG. 2. Intense complete staining of the membrane can be observed. The fluorescence intensity of the membrane areas was quantified and the data for five different breast cancer patients is presented in the following table. HER-2/neu expression of four of the patients was about 2 to 4-fold above the autofluorescence. In one patient the autofluorescence and HER-2/neu signals were similar. There is heterogeneity in HER-2/neu expression across various areas of a tumor. We have observed a 3.3-fold difference between high expression areas and lower expression areas.

Herceptin-Al 532 Staining Of Breast Cancer Tissues From UMM And WR-877 Millisecond Exposure

| | Ave | Percent Standard |
|---|---|---|
| | flu/pix of image* | Reference |
| | Image Number | Image Number |
| Patient | 1 2 3 4 | 1 2 3 4 |
| UMM 1804 111914 A4 | 305, 285, 303 | 15.2, 14.2, 15.2 |
| HER2 Status Unknown | mean is 298 | mean is 14.9 |
| Autofluorescence No | 231, 285, 311 | 11.6, 14.2, 15.6 |
| ABY | mean is 276 | mean is 13.0 |
| UMM 01 505 1295 A9 | 473, 496, 564 | 23.6, 24.8, 28.2 |
| HER2 Status +3 | mean is 511 | mean is 25.5 |
| Autofluorescence No ABY | 151, 147, 135 | 7.6, 6.8, 7.4 |
| | mean is 144 | mean is 7.2 |
| UMM 01 501 2051 C8 | 380, 522, 533, 586 | 19.0, 26.1, 26.7, 29.3 |
| HER2 Status +3 | mean is 505 | mean is 25.3 |
| Autofluorescence No ABY | Not Done | |
| WR 505 3597 B2 Exp 1 | 191, 167, 153, 172 | 9.5, 8.3, 7.7, 8.6 |
| HER2 Status +3 | mean is 171 | mean is 8.5 |
| Autofluorescence No ABY | Not Done | |
| WR 505 3597 B2 | 182, 170, 220 | 9.51, 8.5, 11.0 |
| HER2 Status +3 | mean is 191 | mean is 9.5 |
| Autofluorescence No ABY | 115, 137, 118 | 5.8, 6.8, 5.9 |
| | mean is 123 | mean is 6.2 |
| WR 505 2759 A1 | 136, 135, 163, 148 | 6.8, 6.8, 8.2, 7.4 |
| HER2 Status +3 | mean is 146 | mean is 7.3 |
| Autofluorescence No ABY | 66, 87, 79 | 3.3, 4.4, 4.0 |
| | mean is 77 | mean is 3.9 |

*Three to four areas of each image were analyzed and the average is shown.
ABY indicates labeled monoclonal antibody used for staining.

We have also quantified the expression of estrogen receptor in breast tumor tissue and beta-tubulin isoform III and ERCC-1 (DNA repair enzyme) in colon tumor tissue. The data are presented in the following table. The average fluorescence of the stained tissue versus the autofluorescence (no antibody) was 5.7, 3.6 and 15.8 for ERCC-1, beta-tubulin III and estrogen receptor, respectively.

Quantitative Measurement Of Drug Response Indicators In Tissue Sections Cut From Paraffin Blocks

| | | Average | Flu/Pix | Range | Flu/Pix |
|---|---|---|---|---|---|
| Tissue | Marker | Mean | Std Dev | Max | Min |
| Colon | ERCC-1 | | | | |
| | Area 1 | 1537 | 395 | 3760 | 799 |
| | Area 2 | 1420 | 393 | 3760 | 738 |
| | Area 3 | 1256 | 345 | 3760 | 434 |
| Control | No Aby | | | | |
| | Area 1 | 243 | 28 | 356 | 146 |
| | Area 2 | 242 | 26 | 334 | 120 |
| | Area 3 | 251 | 39 | 410 | 42 |
| Colon | beta-Tub | | | | |
| | Area 1 | 533 | 90 | 876 | 301 |
| | Area 2 | 486 | 89 | 805 | 253 |
| | Area 3 | 485 | 84 | 754 | 186 |
| Control | No Aby | | | | |
| | Area 1 | 136 | 23 | 245 | 73 |
| | Area 2 | 129 | 20 | 215 | 63 |
| | Area 3 | 150 | 28 | 358 | 72 |
| Breast | Estrogen Recp | | | | |
| | Area 1 | 1278 | 307 | 3326 | 639 |
| | Area 2 | 1489 | 269 | 3196 | 840 |
| | Area 3 | 1399 | 318 | 3218 | 844 |
| Control | No Aby | | | | |
| | Area 1 | 73 | 20 | 161 | 18 |
| | Area 2 | 96 | 25 | 340 | 30 |
| | Area 3 | 94 | 21 | 394 | 47 |

### EXAMPLE 5

Staining and numerical measurement of drug response indicators in tissue sections of formalin-fixed tumor embedded in paraffin blocks using breast cancer as an example.

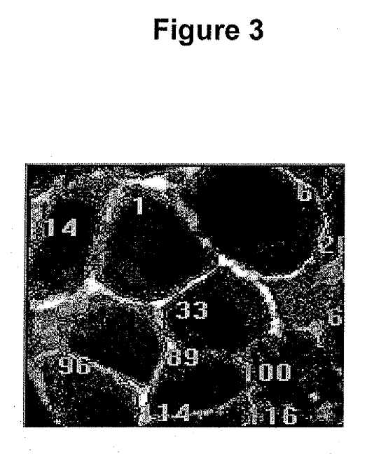

We have established a reproducible fluorescence microscopy procedure for quantifying HER-2/neu receptor expression in breast tumor tissue sections cut from paraffin blocks. Tissue sections mounted on microscope slides are deparaffinized as follows: Place slides in xylene for five minutes, repeat once; Place slides in 95% ethanol for three minutes, repeat once; Place slides in 70% ethanol for three minutes, repeat once; Wash slides in distilled water. Slides are then processed for antigen retrieval by heating in 10 mM citrate, pH 6.0, for 30 minutes at 95 degrees followed by 20 minutes cooling at room temperature. Slides are incubated simultaneously with a Trastuzumab-Alexa 532 conjugate and antipancytokeratin-FITC. Three to four digital images of each tumor section are acquired using an exposure time that yields 2000 fluorescence units with a standard reference. Cells that overexpress HER-2/neu show a very intense fluorescence staining of the complete cellular membrane. Three or four areas of interest (AOI) are examined on each digital image. Histograms displaying quantitative fluorescence data (mean fluorescence per pixel, standard deviation, minimum and maximum values, and the integrated fluorescence intensity) for the AOIs can be generated. This fluorescence data is used to select regions of the stained membrane for quantification of HER-2/neu expressing. FIG. 3 shows the regions on one of the Above AOIs that are selected and outlined for quantification. The quantitative measurement data (area in number of pixels and average fluorescence per pixel, density lum) are shown in the following table.

| | | Average fluorescence |
|---|---|---|
| Object # | Area (pixels) | per pixel |
| 1 | 476 | 203.9727 |
| 6 | 60 | 196.6833 |
| 14 | 215 | 198.4419 |
| 25 | 82 | 194.2927 |
| 30 | 226 | 192.2876 |
| 33 | 1788 | 205.6365 |
| 65 | 51 | 184.9412 |
| 89 | 79 | 202.6456 |
| 96 | 99 | 191.2121 |
| 100 | 52 | 189.9038 |
| 114 | 198 | 208.0404 |
| 116 | 100 | 189.540 |

### EXAMPLE 6

Assessment of heterogeneity in HER-2/neu expression within different areas or section.

Heterogeneity in HER-2/neu expression among the cells in a patient's tumor could determine the overall and duration of response to treatment with Trastuzumab. We have been able to assess this heterogeneity in patient tumor tissue sections utilizing the quantitative assay described above. The data from four AOIs of digital images of tumor tissue for two breast cancer patients are shown in the following table. We observed a 2.1 (patient 1) to 3.3 (patient 2) fold difference between the maximum and minimum fluorescence intensity of the HER-2/neu-stained membranes.

Assessment of Heterogeneity in Her-2Neu Expression within Different Areas of Patient Tumor

| | | | Patient #2 | |
|---|---|---|---|---|
| | | | Ave Fluorescence | |
| | Patient #1 | | Per Pixel | |
| | Mean | Std Dev | Mean | Std Dev |
| | Ave Fluorescence | | | |
| | Per Pixel | | | |
| Area 1 | 473 (25)** | 40 | 380 (22)** | 31 |
| Area 2 | 493 (24) | 34 | 522 (24) | 41 |
| Area 3 | 559 (26) | 37 | 533 (21) | 31 |
| Area 4 | | | 586 (24) | 73 |

| | Autofluorescence | |
|---|---|---|
| | Subtracted | |
| Max Fluorescence/Tumor | 491 | 569 |
| Min Fluorescence/Tumor | 230 | 173 |
| Maximum/Minimum | 2.1 | 3.3 |

**Number of membrane measurements

### EXAMPLE 7

An in vitro system to correlate the cytotoxic action of chemotherapeutic drugs with the expression of mechanistically related Drug Response Indicators.

This example describes an in vitro system that correlates the cytotoxic response of cultured human cancer cell lines to anticancer agents with the level of expression of mechanistically related drug response indicators (DRI) in the cells. Statistical analysis will be performed on this data in order to establish a DRI expression level that may be used to indicate cytotoxic response categorically.

Tumor derived immortal cell lines generally display robust proliferation and fill a need for functional cancer cell model systems. Cancer cell lines have been utilized for prediction of responses to anticancer drugs with some degree of success, indicating that chemosensitivity profiles might be useful to develop systems for the prediction of drug efficacy.

In the present example, DRI are quantitated in a number of breast cancer derived cell lines, utilizing monoclonal antibodies linked With fluorescent dyes as probes. The level of DRI expression is expressed as a digital value normalized to a readily available fluorescence standard to allow for inter-day and inter-laboratory comparison of data. All drugs will have potential cellular targets that are mechanistically related to the mode of action.

Breast cancer cell lines were fixed and stained on microscope slides by simultaneous incubation with herceptin (trastuzumab, anti-HER-2/neu receptor) conjugated with Alexa 532 or anti-ER conjugated with Alexa 594 or anti-TUB III conjugated with Alexa 647 and anti-cytokeratin conjugated with FITC. Digital images of the FITC signal (470 nm/497 nm/522 nm), Alexa 647 signal (630 nm/649 nm/667 nm), Alexa 594 signal (581 nm/593 nm/617 nm) and the Alexa 532 signal (546 nm/557 nm/567 nm) were acquired at the appropriate exposure times (which yields a value of 2000 with the fluorescence standard) and analyzed to determine the average fluorescence per pixel in each ROI (cancer cell). The spatial area of each ROI was determined from the cytokeratin fluorescence, which is very strong. The outlines are saved, recalled and overlaid on an Alexa 532 image, an Alexa 594 image or an Alexa 647 image of an identical field of cells. The software generates a table showing the area and average fluorescence per pixel of each ROI.

For experimental conditions, cell suspensions containing 10⁴ viable cells were plated into 96 well plates in 100 μl of media, and allowed to attach for 24 hours at 37° C. in a 5% CO₂ atmosphere. After this incubation period, the cells were exposed to the drug at the designated doses, which were based on peak plasma concentration (PPC), as determined by published pharmacokinetic analyses. In the case of Tamoxifen, cells were grown for 72 hours until confluent. Media containing 0.5% FCS was then added to each well in order to maximize ER expression. Control wells contained 100 μl of the appropriate media and were treated identically to the test wells. At 72 hours post treatment, the plates were subjected to WST-8 analysis. WST-8 is a tetrazolium salt that is bioreduced by cellular dehydrogenases to yield a colored formazan product. The amount of the formazan product is directly proportional to the number of living cells. Formazan based assays have successfully been utilized for chemosensitivity testing. The absorbance of each well was measured at 460 nm using a Biotek microplate reader. For each concentration of drugs the mean absorbance±the SE was calculated. Results are expressed as % inhibition of growth compared with drug concentration. IC₅₀ values will be determined using the median effect plot of (log fa/log fu) vs log C where fu=fraction unaffected, fa=fraction affected and C=drug concentration.

The biological response of the various breast cancer cell lines to chemotherapeutic drugs corresponds well with expression of the related DRI. Initial studies were conducted with two well-characterized DRI's, HER-2/neu and the estrogen receptor (ER), in order to confirm the model system. The HER-2/neu oncogene encodes a transmembrane tyrosine kinase receptor with extensive homology to the epidermal growth factor receptor, HER-2/neu overexpression results in increased sensitivity to Herceptin (trastuzumab) therapy. Another effective targeted therapy for breast cancer is tamoxifen, which binds to the estrogen receptor on the surface of cancer cells and blocks the effects of estrogen on cell growth. ER is known to have a significant predictive value in determining sensitivity to Tamoxifen therapy.

Cell lines exhibiting the highest levels of fluorescently labeled Herceptin binding on their surface showed the greatest response to treatment with Herceptin. These particular cell lines, HCC2218 and SKBR-3, displayed a dose-related inhibition of proliferation, with responses at concentrations as low as 0.032 and 0.125 mg/ml respectively. In contrast, as shown in the following table, in breast cell lines exhibiting low levels of Herceptin binding, proliferation was either unperturbed, or a response was elicited only with high doses of the drug.

Comparison of Biological Response with DRI Expression Levels in Cultured Breast Cancer Cells.—Her-2/neu/Herceptin

| | | | Mean | | Median | | |
|---|---|---|---|---|---|---|---|
| | | | Herceptin | | Herceptin | Range Herceptin | # Cells |
| CELLS | IC50-1 | IC50-2 | Binding** | SD | Binding** | Binding** | analyzed |
| HCC 2218 | 0.06 | 0.12 | 53.83 | 16.54 | 51.05 | 31.9-87.3 | 48 |
| SKBR 3 | 0.30 | 0.65 | 21.45 | 6.60 | 20.3 | 1.7-37.1 | 52 |
| MCF-7 | 1.25 | 1.36 | 3.11 | 1.16 | 3.1 | 1.12-5.8 | 57 |
| T47D | 2.0 | 2.0 | 3.55 | 1.46 | 3.45 | 0.85-7.7 | 59 |
| HCC 38 | NR* | NR* | 3.86 | 1.67 | 3.45 | 0.9-8.95 | 37 |
| HCC 202 | NR* | NR* | 2.96 | 0.82 | 2.85 | 1.6-4.75 | 54 |

Notes:
*NR—No response to Herceptin treatment up to 2 mg/ml
**Average fluorescence/pixel relative to a reference standard that yields 2000 units when subjected to a defined exposure time

In a similar fashion, a cell line (MCF-7) displaying a high ER expression showed a high sensitivity to treatment with Tamoxifen. In contrast cell lines displaying a lesser degree of ER binding (SKBR-3, MDA-MB 231) displayed a much less sensitive response to treatment with Tamoxifen as seen in the following table,

Comparison of Biological Response with DRI Expression Levels in Cultured Breast Cancer Cells.—Estrogen Receptor/Tamoxifen

| | IC50-1 | IC50-2 | ER | | ER | | # Cells |
|---|---|---|---|---|---|---|---|
| Cell type | μg/ml | μg/ml | Mean** | SD | median** | ER/Cell Range* | analyzed |
| mcf-7 | 9.8 | 13 | 57.0 | 12 | 60.0 | 35-91 | 45 |
| T47D | 28.3 | 21.8 | 49.0 | 16 | 45.0 | 16-80 | 45 |
| SKBR3 | 41.6 | 35 | 43.0 | 10 | 43.0 | 25-61 | 32 |
| MDA- | 42.5 | 42.5 | 23.0 | 7 | 22.0 | 9-41 | 61 |
| MB231 | | | | | | | |
| HCC 202 | NR* | NR** | 25.0 | 8 | 23.0 | 13-42 | 33 |
| HCC2218 | NR* | NR** | 29.7 | 12 | 25.7 | 18-88 | 34 |
| HCC38 | NR* | NR** | 32.1 | 7 | 31.4 | 22-53 | 30 |

Notes:
*NR—No response to Tamoxifen treatment up to 50 μg/ml
**Average fluorescence/pixel relative to a reference standard that yields 2000 units when subjected to a defined exposure time.

These experiments were extended to consider the correlation between beta-tubulin III (TUB III) expression and the biological response to the anticancer drugs, paclitaxel (PTX) and docetaxel (DTX). These drugs bind to TUB III and exert their growth inhibitory effects through the stabilization of the microtubule. It is speculated that the antitumor action of these drugs can be modified by the expression level of TUB III in several human cancers, including breast tumors.

Comparison of Biological Response with DRI Expression Levels in Cultured Breast Cancer Cells.—Paclitaxel/Beta-tubulin III

| | IC50-1 | IC50-2 | TUB III | | | Cell Range | # Cells |
|---|---|---|---|---|---|---|---|
| Cell type | μg/ml | μg/ml | Mean** | SD | TUB III median* | TUB III | analyzed |
| T47D | 3 | 3.6 | 2.22 | 0.52 | 2.18 | 1.4-3.4 | 19 |
| MCF-7 | 6 | 7.1 | 3.58 | 1.03 | 3.42 | 2.1-6.3 | 39 |
| SKBR-3 | 10 | 8.5 | 4.64 | 2.07 | 4.01 | 2.6-14.2 | 19 |
| HCC2218 | NR* | NR* | 31.79 | 9.61 | 28.1 | 20.6-60.4 | 30 |
| HCC 202 | NR* | NR* | 34.49 | 10.5 | 32.53 | 20.7-80.7 | 78 |
| HCC 38 | NR* | NR* | 49.19 | 15.95 | 47.16 | 30.8-98.4 | 56 |

Notes:
*NR—No response to Paclitaxel treatment up to 50 ug/ml
**Average fluorescence/pixel relative to a reference standard that yields 2000 units when subjected to a defined exposure time

Comparison of Biological Response with DRI Expression Levels in Cultured Breast Cancer Cells.—Docetaxel/Beta-tubulin III

| | IC50-1 | IC50-2 | TUB III | | | cell range | # Cells |
|---|---|---|---|---|---|---|---|
| Cell type | μg/ml | μg/ml | Mean** | SD | TUB III Median* | TUB III | analyzed |
| MCF-7 | 1.0 | 0.8 | 2.2 | 0.5 | 2.2 | 1.4-3.4 | 19 |
| T47D | 5.5 | 4.5 | 3.6 | 1.0 | 3.4 | 2.1-6.3 | 39 |
| SKBR3 | 6.6 | 12.5 | 4.6 | 2.1 | 4.0 | 2.6-14.2 | 19 |
| HCC 2218 | 25.0 | 18.0 | 31.8 | 9.6 | 28.1 | 20.6-60.4 | 30 |
| HCC202 | 25.5 | 22.0 | 34.5 | 10.5 | 32.5 | 20.7-80.7 | 78 |
| HCC38 | 58.0 | 59.9 | 49.2 | 16.0 | 47.2 | 30.8-98.4 | 56 |

Notes:
**Average fluorescence/pixel relative to a reference standard that yields 2000 units when subjected to a defined exposure time

As in the case of Herceptin and Tamoxifen, the biological response of the various breast cancer cell lines to PTX and DTX corresponds well with expression of TUB III. Cell lines displaying a low level of TUB III binding (T47D, MCF-7, SKBR-3) were sensitive to treatment with both PTX and DTX. In contrast, cell lines displaying a higher level of TUB III (HCC 2218, HCC 38) showed a response only to higher doses of DTX. These cell lines also displayed a response to PTX, but did not express an IC₅₀ value. One cell line (HCC 202) proved resistant to both DTX and PTX treatment.

In another example, the expression of thymidylate synthase (TS) was correlated with the response of breast cancer cells to the chemotherapeutic drug 5-fluorouracil (5-FU). TS is a key enzyme in DNA biosynthesis and has been postulated to play a major role in predicting the response to 5-FU based chemotherapy. Experiments were conducted to examine this relationship. Experimental conditions and analysis methods were similar to those used with Herceptin. Doses ranged from 5-300 μg/ml.

Comparison of Biological Response with DRI Expression Levels in Cultured Breast Cancer Cells.—5 Fluorouracil/Thymidylate Synthase

| CELL | IC50-1 | IC50-2 | TS | | | Cell Range | # Cells |
|---|---|---|---|---|---|---|---|
| LINE | FU | FU | Mean** | SD** | TS Median** | TS | analyzed |
| T47D | 8.64 | 6.72 | 25.2 | 7.6 | 23.2 | 18.1-56.6 | 77 |
| MCF-7 | 10.0 | 15.2 | 20.4 | 2.1 | 20 | 15.5-23.4 | 14 |
| SKBR-3 | 23.3 | 33.7 | 18.2 | 3.3 | 18.2 | 11.6-25.5 | 46 |
| HCC 202 | 181.3 | 162.5 | 15.1 | 2.2 | 14.6 | 11.0-20.0 | 24 |
| HCC 38 | 215.7 | ND | 11.9 | 2.3 | 12.1 | 9.1-16.5 | 33 |
| HCC 2218 | 246.1 | ND | 10.6 | 1.9 | 10.4 | 7.3-15.3 | 49 |

Notes:
**Average fluorescence/pixel relative to a reference standard that yields 2000 units when subjected to a defined exposure time.
ND = not determined

The biological response of the various breast cancer cell lines to 5-FU corresponds well with expression TS. Cell lines responding to lower doses of 5-FU (T47D, MCF-7 and SKBR-3) displayed higher expression of TS than those cell lines that responded only to higher doses (HCC 38, HCC 202, HCC 2218). The correlation of the rank of response to 5-FU with TS expression was statistically significant as determined by Pearson's Correlation Coefficient as seen below.

The DRI may be statistically correlated with the inhibition of in vitro cell growth by mechanistically related drugs using Pearsons Correlation Coefficient. This method measures the strength of the linear relationship between two variables. Pearson's Correlation Coefficient is usually signified by r (rho), and can take on the values from −1.0 to 1.0° where −1.0 is a perfect negative (inverse) correlation, 0.0 is no correlation, and 1.0 is a perfect positive correlation. Pearson's Correlation Coefficient may be calculated using the formula below:

$$r = \frac{\sum XY - \frac{\sum X \sum Y}{N}}{\sqrt{( \sum {X}^{2} - \frac{{( \sum X )}^{2}}{N} ) ( \sum {Y}^{2} - \frac{{( \sum Y )}^{2}}{N} )}}$$

Pearson's Coefficient Correlation analysis indicated a strong and significant statistical correlation between IC₅₀ values and DRI expression as seen in the following table.

Correlation of Biological Response and DRI Expression

| | | PEARSON'S | |
|---|---|---|---|
| DRUG | DRI | COEFFICIENT | p Value |
| Herceptin | Her 2/neu | −0.941 | 0.0005 |
| Tamoxifen | Estrogen Receptor | −0.976 | 0.02 |
| Docetaxel | Beta Tubulin III | +0.978 | .001 |
| Paclitaxel | Beta Tubulin III | +0.990 | .001 |
| 5-Fluorouracil | Thymidylate | −0.943 | .002 |
| | Synthase | | |

This correlation data will be analyzed in order to further identify a DRI expression cut-off point through a simple cluster analysis or change point analysis to determine if there is a DRI expression level that may be used to indicate biological response categorically.

### EXAMPLE 8

DRI Index system based on paraffin embedded cultured cell standard.

In order to index the DRI expression of human tumor tissue preserved in paraffin blocks, it is necessary to establish a control standard that reflects the influences of tissue fixation and processing. A number of laboratories have utilized cell lines that display variable expression of the targeted antigen for this purpose. These cell lines are fixed, processed and analyzed in a fashion identical to the clinical sample. This technology has been successfully applied to the standardization of HER-2/neu assay sensitivity, immunocytochemical analysis of estrogen receptor, DNA ploidy analysis and quality control of the proliferation marker MIB-1.

Cultured human breast cancer cells were harvested from the same batch used for the cultured human tumor cell system. These cells were then embedded in agar plugs and processed to paraffin. Slides were prepared with 4 um sections, fixed and stained with HER-2-Alexa 532 (546 nm/557 nm/567 nm). Samples were then subjected to fluorescence microscopic analysis as described above. Results (following table) were very similar to those observed in the cultured tumor cell standard system. Both HCC 2218 and SKBR-3 displayed high Herceptin binding values, while MCF-7 and HCC 38 displayed lower values. Comparison of the IC₅₀, derived by the exposure of these cells to Herceptin, and Herceptin binding resulted in a Pearson's correlation coefficient of −0.9, again very similar to that observed in the cultured tumor cell standard system. This model indicates that the paraffin embedded cultured cell standard may serve as a viable internal standard for indexing the biological response of paraffin embedded human cancer tissue.

Comparison of Biological Response with DRI Expression Levels cultured human breast cancer cells Trastuzumab/Trastuzumab—binding

| | | |
|---|---|---|
| | MAB binding | # Cells |
| IC50 | Mean** | analyzed |

| CELLS | mg/ml | Fresh | paraffin | Fresh | paraffin |
|---|---|---|---|---|---|
| HCC 2218 | 0.06 | 53.8 | 31.3 | 48 | 59 |
| SKBR 3 | 0.30 | 21.5 | 25.1 | 52 | 62 |
| MCF-7 | 1.25 | 3.1 | 8.11 | 57 | 39 |
| T47D | 2.00 | 3.5 | ND | 59 | ND |
| HCC 38 | NR* | 3.9 | 5.21 | 37 | 48 |
| HCC 202 | NR* | 3.0 | ND | 54 | ND |

ND = not determine
NR = No Response

### EXAMPLE 9

A statistical analysis of the correlation of DRI expression with the response to anticancer drugs for the construction of a DRI reference range index.

The rationale and the approach for construction of the DRI reference range of tumor response is based on: (1) in vitro indexing system; (2) paraffin block in vitro indexing system. The success in the construction of these two in vitro systems provides the confidence that the cytotoxic response of tumor cells can be correlated statistically to DRI measurement after paraffin embedding.

The purpose of this index is to compute a probability of resistance to a drug at different levels of DRI expression of an individual patient's tumors This reference range can then be consulted by the attending physician for anticancer drug prescription to a given cancer patient with a certain level of DRI expression.

We will use statistical regression approach to model the inhibition of in vitro cell growth by each drug with the level of DRI expression pertinent to that drug. This method will be applied to both the cultured tumor cell standard and the paraffin embedded cultured cell standard. The outcome variable is the logarithm of IC₅₀ determined from the dose response curve for cell line and the independent variable is the DRI level. The log IC₅₀ is used because of our preliminary data and those in the literature. In this proposed study, with 13 cell lines, we would have adequate power (80% chance) to show that the true correlation is at least 60% at a significance level of 5%. We set the alternative hypothesis for the correlation to be 0.90 in the calculation, which is plausible since the Pearson correlation is observed to above 0.90 in the above cited experimental examples.

We will analyze this correlation data further through a simple cluster analysis or change point analysis. This analysis will determine whether there is a DRI expression level that may be used to indicate biological response probability categorically.

### EXAMPLE 10

The three molecular diagnostic tests in providing information for management of the disease for individual cancer patients.

Drug Response Indicators Test (DRIT)

Description
1) 6-8 cellular biomarkers, expressed in cells of primary tumors (embedded in paraffin blocks) and related to both tumor resistance and targeted chemotherapy are stained with fluorescently labeled monoclonal antibodies (fMAb). The expression level of these markers can be quantified utilizing a computer-assisted fluorescence microscopy system and used for targeted chemotherapy.
2) The numerical fluorescent measurements of these biomarkers are normalized to a commercially available fluorescence standard to allow for comparison of interday-interlaboratory data.
3) In vitro calibration systems were established consisting of cultured cancer cells from cell lines of a cancer type (such as breast or lung, etc.) with varying degrees of resistance to treatment with a targeted therapy. A significant correlation between the extent of resistance (as measured as IC₅₀) and the expression of the biomarkers (fluorescence per pixel) has been established by Pearson's coefficient. A statistical regression approach will be used to model the inhibition of in vitro cell growth by each drug with the level of DRI expression pertinent to that drug. The outcome variable is the logarithm of IC₅₀ determined from the dose response curve for cell line and the independent variable is the DRI level. Simple cluster analysis or change point analysis will be employed to determine if there is a DRI expression level that may be used to indicate the probabilities of unfavorable biological response.
4) These cell lines were also fixed and embedded in paraffin blocks in the same manner as the primary tumor. Sections of these cells on slide are stained and measured as described above. A correlation is again established between the IC₅₀ of these cells and the expression of the corresponding biomarkers.
5) The above correlative studies (in cancer cell lines) between IC₅₀ and the expression of corresponding biomarkers were used to establish DRI Indices for the biomarkers. Such indices can be used for correlating the biomarker expression of a tumor and the extent of resistance of this tumor to treatment with a mechanistically related drug. The DRI indices may be used to delineate probabilities of resistance (treatment failure).
6) These laboratory studies will be confirmed and modified by retrospective clinical correlative studies and prospective clinical correlative studies, so that the DRI index and its reference range of probabilities will be based on clinical data.
7) The tests are performed on 4 micron thick paraffin block sections of the primary tumor (6 slides required for duplicate measurements) and it takes 2 days. Fixed tumor cells, not embedded, can also be readily tested.

Application
1) A confidential report of the test will provide DRI indices and the statistically computed probability of the resistance of an individual patient's tumor to 5-6 FDA approved, widely used chemotherapeutic drugs included in the guidelines of the National Comprehensive Cancer Network (NCCN) made available by the National Cancer Society. The physician can make informed decisions based upon the test results about the most appropriate regimen for this patient.

HER-tax Test

Description
1) The HER-tax Test is to be applied to breast cancer patients who are HER-2/neu receptor positive (overexpression shown by IHC 3+ or FISH+).
2) The test is performed on primary tumors embedded in paraffin blocks in a similar fashion to the DRIT except staining for HER-2/neu receptor, (with fluorescently labeled Herceptin), PTEN and for β-tubulin III (the DRI for taxanes).
3) The objective is to screen for tumors in patients who display HER-2/neu overexpression in the low range (but still IHC 3+), and who also display a low value of PTEN expression and a high value of β-tubulin III expression. This patient population's tumors could be resistant to both Herceptin and taxane. A probability for resistance will be provided.
4) The heterogeneity of cancer cells in the tumor with respect to expression of HER-2/neu and β-tubulin III will be measured. The degree of heterogeneity could be related to duration of favorable response to the Herceptin-taxanes regimen.

Application
1) Since only 40-50% of the HER-2/neu positive patients will respond favorably to the Herceptin-taxane regimen for a limited duration (most likely not more than one year), this HER-tax test Will select the resistant patients and predict the likely duration of favorable response for patients under treatment. Herceptin is a reasonably expensive drug and has cardiac toxicity.

Circulating Cancer Cell Test (CCCT) for Breast Cancer Patients

Description
1) 20 ml of the patient's venous blood is collected and the circulating cancer cells (CCC) are enriched by removal of the normal blood cells through density gradient centrifugation and magnetic cell sorting. These cancer cells are collected via negative selection procedures, placed on microscope slides, and then stained with fluorescent monoclonal antibodies and enumerated utilizing a computerized fluorescence microscopy system.
2) The test is carried out within 24 hours after the blood sample collection. The sample can be sent by expressed mail in a special shipping container verified by CCC Diagnostics.
3) Drug response biomarkers expressed in CCC can be characterized.
4) The number of the CCC found in metastatic cancer (Stage IV) is higher than that in Stages I, II, and III cancer. High numbers of CCC are statistically related to poor prognosis and poor response to drug treatment.
5) In a proposed drug treatment study, the enumeration of CCC in a test before the treatment and 2-3 tests during and after treatment (2-3 month time interval), may reflect the treatment impact. If the number of CCC remains high after treatment, the drug is likely not effective, and if the number of CCC becomes much lower after treatment, the drug is likely to be effective. This preliminary finding will be tested clinically. The technique of finding CCC in the blood has been developed for 7 cancers (prostate, lung, gastric, pancreatic, liver, colon, and breast),

Application
1) For Stage III or Stage IV breast cancer patients, a CCCT can be done before treatment to evaluate the prognosis and 2-3 CCCT can be done following the treatment to note the response. The results can be obtained before the imaging examination α-rays and CT's) of the patient.
2) When the drug resistance of the tumor may occur, CCCT can be done to measure the change of the biomarker status in CCC in order to make a new choice for the next drug treatment. These results may be correlated with in vitro calibration systems consisting of cultured cancer cells from cell lines of a cancer type (such as breast or lung, etc.) with varying degrees of resistance to the treatment of a targeted therapy. The correlation will be based on the expression of designated DRI's and the response of the cultured cells to drug treatment.
3) While the data on the number of CCC may not replace the imaging examination, the CCCT is faster and provides insight into the changing of drug resistance characteristics of the metastatic tumors. Characterization of the resistance of tumors with respect to biomarkers can not be done by imaging approach.

### EXAMPLE 11

PAC requires three strategic steps, Step I: Quantitative measurement of several targets simultaneously in the tumor. These targets, termed drug response indicators (DRI), can be evaluated by immunofluorescence utilizing labeled monoclonal antibodies and a computerized fluorescence microscope. Numerical values can be derived and normalized to a fluorescent reference standard for comparison.

Step II: Establishment of statistically significant correlation of DRI expression with the cytotoxic response of tumor cells to a related drug. An in vitro indexing system was established to correlate the cytotoxic effect of each drug to the corresponding DRI measurements. Seven breast cancer (BC) cell lines with different sensitivities to various anticancer drugs were utilized. The effect of tamoxifen, paclitaxel, trastuzumab, and doxorubicin was correlated with the DRI expression for each drug in these cell lines. The DRI are estrogen receptor, beta tubulin III, HER-2/neu and topoisomerase II, respectively. Pearson rank correlation coefficients are found ranging from 0.77 to 1 and the p values ranging from 0.005 to 0.02.

Step III: Technology for obtaining cancer cells from individual cancer patients. For metastatic tumors, circulating cancer cells (CCC) from peripheral blood were obtained using a negative selection procedure (Cancer 2000: Vol. 88, no. 12, p.

2787), enumerated, and stained with labeled trastuzumab in order to quantify the HER-2/neu expression. One hundred and one BC patients were studied and 402 blood samples drawn; median number of samples drawn per patient was 4 (1-7). CCC are related to distant metastasis; 88% of Stage IV patients have CCC at some point during sampling. CCC numbers ranged from 1-1283 per sample.

Twenty patients had four or more of CCC to test for HER-2/neu expression and also had available tumor tissue data. In 18 patients CCC and primary tumor data concurred (90%) with 6 HER-2/neu positive and 12 HER-2/neu negative. One patient was HER-2/neu negative in tumor tissue but HER-2/neu positive in CCC. One patient was identified as HER-2/neu positive in tumor tissue and HER-2/neu negative in CCC.

Therapy (trastuzumab)-diagnostics (HER-2/neu expression) coupling was the first model approved by FDA for PAC for BC. However fewer than 30% of HER-2 positive metastatic BC patients respond to trastuzumab as single agent therapy though HER-2 negative patients are significantly less responsive. Improvement of DRI measurements in BC tumor tissue and CCC may lead to more predictable treatment outcomes. DRI measurement from section slides cut from primary tumor tissues of BC patients will also be reported.

### EXAMPLE 12

Quantification of DRI in formalin-fixed paraffin-embedded (FFPE) tissue from breast, colon and lung cancer patients

The preparation and incubation of FFPE tissue sections mounted on microscope slides with fluorescently-labeled monoclonal antibodies is described in Examples 1 & 4. Digital images of the same field of cells were acquired with filter cubes which discriminate DAPI, FITC, Alexa 532, Alexa 594 and Alexa 647 fluorescence utilizing exposure times determined from standard curves as described in Example 2. Images of five different areas from each tumor tissue section were obtained and stored. Background images were acquired with each filter cube at the appropriate exposure times utilizing a slide that does not contain any tissue. To evaluate tissue autofluorescence, images were also acquired of a serial section from each tumor, incubated only with anti-pan cytokeratin-FITC, with each of the above filter cubes and processed exactly as the test sample.

To quantify DRI fluorescence intensity, the images were first flat-fielded by subtracting the appropriate background image from the DRI image. This eliminates any variations in the illumination field. The average fluorescence per pixel (F/P) of the background image (obtained from the image histogram) is then subtracted from the flat-fielded image. The processed DRI image is examined interactively and the area with the brightest fluorescence is outlined (usually 50 to 100 cells). This area of interest (AOI) is duplicated and saved as a cropped image. The saved cropped image is recalled and set on an image of the same field of cells acquired with the FITC filter to ensure that all the cells in the AOI are cytokeratin-positive epithelial cells. The fluorescence signals in each cropped DRI image is analyzed by Image-Pro software to select the intensities that separate the fluorescent objects (cells or cell clusters) from the background. Image-Pro will then outline, count and present quantitative data on the objects in the cropped DRI image. The data is exported to Excel and the mean of the average F/P for the objects in each of the five images from each tumor is calculated and normalized to the reference standard. The autofluorescence of a serial section of the same tumor is calculated for each filter cube utilizing the exact same procedure used for the test slide. The average fluorescence per pixel of each DRI image (five per tumor, total of 250 to 500 cells) minus tissue autofluorescence is reported as percent of the standard reference.

DRI results on FFPE tissue of six breast cancer patients are presented in the first Table below. Estrogen receptor (ER), beta-tubulin isoform III (TUB) thymidylate synthase (TS), topoisomerase II (TOP), ribonucleotide reductase (RR), HER-2/neu (HER) and excision repair cross complementary-1 enzyme (ERCC-1) were quantified. The average F/P (normalized to reference standard) of the selected objects in five cropped images of each DRI measured in six different patients is presented along with the mean of the five values (shown in bold type) and the coefficient of variation (CV) of the five values (shown in parenthesis). The CV values for five DRI images from the six patients ranged from 8.9 (TS of patient 39) to 37.4 (ER of patient 39); in five of 18 measurements, the CV did not apply because of very low DRI values. When duplicate serial sections of the same patient were analyzed, the DRI values were in the same relative range as seen in the following table.

Breast Cancer Study

| Patient # | ER | TUB III | TS | TOPO | RR | HER-2 | ERCC-1 |
|---|---|---|---|---|---|---|---|
| 3 | 23, 17, 18, | 9, 24, 16, | | | | | |
| | 29, 19 | 18, 16 | | | | | |
| | 21 | 16 | | | | | |
| | (23.2) | (32.3) | | | | | |
| 20 | | 11, 13, 9, | | | 34, 33, | 0..5, 1.8, | |
| | | 10, 18 | | | 33, 39, 40 | 0.4, 0, 1.8 | |
| | | 12 | | | 36 | 0.9 | |
| | | (29.2) | | | (9.6) | (na) | |
| 39 | | 7, 5, 9, 13, 6 | 48, 47, 42, | | | | |
| | | | 34, 34 | | | | |
| | | 11 | 41 | | | | |
| | | (24) | (16.5) | | | | |
| | | 9, 19, 9, 9, | 52, 55, 50, | | | | |
| | | 12* | 62, 59 | | | | |
| | | 12 | 56 | | | | |
| | | (37.4) | (8.9) | | | | |
| 43 | 0.2, 0, 0, | 31, 29, 35, | | | | 0, 0, 0, 0, 0 | 72, 52, 63, |
| | 0.4, 0 | 40, 44* | | | | | 84, 62 |
| | 0.1 | 36 | | | | 0 | 67 |
| | (na) | (17.4) | | | | (na) | (18.5) |
| 70 | 30, 26, 28 | | | 5, 5, 7, 4, 5 | | | |
| | 24, 33 | | | | | | |
| | 28 | | | 5 | | | |
| | (28.2) | | | (21.1) | | | |
| | | | | 3, 3, 2, 4 | | | |
| | | | | 3 | | | |
| | | | | (27.2) | | | |
| 75 | | 12, 8, 3, 2, 2 | | | | 9, 5, 0, 15, 0 | |
| | | 5 (na) | | | | 6 (na) | |

The DRI values are presented as a percent of a standard reference (fluorescent microsphere), *Second antibody used, goat anti-mouse IgG-Alexa 532, Number in parenthesis is the coefficient of variation for the DRI values measured in five different area of tumor tissue.

Slides of FFPE tissue of breast, lung and colon cancer patients were obtained from the NCI Cooperative Human Tissue Network (CHTN), Mid-Atlantic Division. The Table below shows DRI measurements for the three different types of cancer patients and also compares ERCC-1 expression values of serial sections from the same patient obtained by two different operators on two different microscopes. The DRI values measured in five cropped images from different areas of the tumor tissue is shown along with the coefficient of variation. The results demonstrate that these four DRI can be measured in the epithelial cells (positive cytokeratin staining) of FFPE tissue from the three cancer types and that similar data was obtained with DRI measurements by two technicians and on two different microscopes. It should be noted that each DRI measurement will be correlated with patients' response to a related anti-cancer drug in order to evaluate the potential of this assay for use by physicians in choosing the appropriate drugs for an individual patient. Such information will be obtained initially in a retrospective study followed by a prospective clinical trial.

Comparison Of DRI Quantification in Breast, Colon and Lung Tissue Sections

| | Breast MAD04- | Lung MAD02- | Colon MAD04- |
|---|---|---|---|
| DRI- slides A, B, D | 00047T | 00394T | 00458 T |
| Date-microscope- | Five Images-% std | Five images-% std | Five images-% std |
| tech | ref | ref | ref |
| TS-A 8/14 L6 SL | 65, 43, 66, 80, 80 | 14, 38, 40, 12, 42 | 47, 59, 77, 58, 70 |
| | 65 CV 22.1 | 29 CV 47.2 | 62 CV 18.2 |
| TS-D 8/22 L5 DZ | | | 52, 67, 53, 79, 69 |
| | | | 64 CV 17.6 |
| ERCC-A 8/14 L6 | 144, 134, 225, 287, | 76, 109, 72, 87, 18 | 75, 76, 109, 77, 96 |
| SL | 85 | 72 CV 45.2 | 86 CV 17.5 |
| | 174 CV 45.8 | | |
| ERCC-D 8/24 L5 | 206, 139, 194, 238, | | |
| DZ | 209 | | |
| | 197 CV 18.4 | | |
| ERCC-D 8/24 L5 | 83, 76, 102, 97, 103 | | |
| SL | 90 CV 13.0 | | |
| ERCC-D 8/24 L6 | 116, 119, 109, 116, | | |
| DZ | 134 | | |
| | 119 CV 7.8 | | |
| ERCC-D 8/24 L6 | 89, 108, 90, 82, 63 | | |
| SL | 86 CV 18.1 | | |
| ERCC-D 8/22 L5 | | | 112, 147, 148, |
| SL | | | 157, 132 |
| | | | 139 CV 12.5 |
| RR-B 8/18 L5 DZ | 11, 10, 10, 20, 10 | 7, 11, 7, 7, 8 | 10, 12, 6, 10, 16 |
| | 11 CV 7.5 | 8 CV 15.5 | 11 CV 11.3 |
| RR-D 9/5 L5 DZ | | 7, 10, 10, 7, 9 | |
| | | 9 CV 12.5 | |
| TUB-B 8/18 L5 DZ | 19, 19, 20, 23, 10 | 8, 13, 7, 7, 11 | 7, 9, 6, 9, 14 |
| | 18 CV 24.7 | 9 CV 25.1 | 9 CV 29.9 |

CV is the coefiicient of variation for the DRI values measured in five different areas of tumor tissue. DRI values are expressed as percent of standard reference with mean shown in bold type.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims. All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method of selecting a chemotherapeutic agent for treatment of cancer for an individual cancer patient, comprising
selecting a panel of government approved chemotherapeutic agents for treatment of the type of cancer suffered by this patient;
obtaining a tumor cell sample from the patient;
determining expression of a panel of drug response indicators corresponding to the panel of chemotherapeutic agents using antibodies in at least one cell of the tumor cell sample; and
selecting a chemotherapeutic agent based on the expression of the drug response indicators.

2. A method according to claim 1, wherein the antibodies are fluorescently labeled.

3. A method according to claim 1, wherein the drug response indicators are cellular components related to the mechanism of action of chemotherapeutic agents.

4. A method according to claim 1, wherein the antibodies are fluorescently labeled with different fluorescent dyes having different excitation and nonoverlapping emission spectra.

5. A method according to claim 1, wherein determining comprises quantifying the drug response indicators.

6. A method according to claim 5, wherein quantifying comprises comparison of fluorescent intensity against a reference standard.

7. A method according to claim 5, wherein determining comprises quantifying at least 5 drug response indicators in one test.

8. A method according to claim 5, wherein determining comprises correlating the quantity of expression of a plurality of drug response indicators in the sample to a quantity of the same drug response indicators in cells of known response to chemotherapeutic agents that act through the drug response indicators.

9. A method according to claim 8, wherein the quantity of expression of at least 5 drug response indicators in a test is compared.

10. A method according to claim 1, wherein the tumor cell sample is selected from the group consisting of circulating cancer cells, a sample obtained from a lymph node adjacent to a primary tumor, a sample from a primary tumor, and a sample from a primary tumor fixed and embedded in paraffin blocks.

11. A method according to claim 1, wherein the chemotherapeutic agent is selected from a group consisting of carboplatin, cisplatin, oxaliplatin, docetaxel, paclitaxel, taxol, vinorelbine, vinca alkaloids, 5-fluouracil related drugs, xeloda, gemcitabine, anthracycline, irinotecan.

12. A method according to claim 1, wherein the chemotherapeutic agent is selected from a group consisting of humanized monoclonal antibodies, trastuzumab (herceptin), cetuximab (erbitux), and beracizumab (avastin).

13. A method according to claim 1, wherein at least one drug response indicator is ERCC1 and the chemotherapeutic agent is selected from the group consisting of Carboplatin, Cisplatin and Oxaloplatin.

14. A method according to claim 1, wherein at least one drug response indicator is beta-tubulin III isoform and the chemotherapeutic agent is selected from the group consisting of Docetaxel, Paclitaxel, Taxane, and Vinorelbine.

15. A method according to claim 1, wherein at least one drug response indicator is Thymidylate Synthase and the chemotherapeutic agent is selected from the group consisting of 5-FU related drugs, Leucovorin, Pemetrexel and Xeloda.

16. A method according to claim 1, wherein at least one drug response indicator is Topoisomerase II and the chemotherapeutic agent is selected from the group consisting of Anthracycline, Doxorubicin, and Epirubicin.

17. A method according to claim 1, wherein at least one drug response indicator is Topoisomerase 1 and the chemotherapeutic agent is Irinotecan.

18. A method according to claim 1, wherein at least one drug response indicator is ribonuclease reductase and the chemotherapeutic agent is Gemcitabine.

19. A method according to claim 1, wherein determining comprises conducting a Drug Response Indicator Test for one or more cancers selected from a group consisting of breast cancer, lung cancer, colon cancer, gastric cancer, pancreatic cancer, and esophageal cancer, wherein the Drug Response Indicator Test provides tumor resistance/sensitivity data for all chemotherapeutic agents listed in the guidelines of the National Comprehensive Cancer Network Treatment for Patients for the type of cancer diagnosed.

20. A method according to claim 19, wherein the tumor resistance/sensitivity data can be interpreted by a percentage probability of drug response failure for a tested individual patient based on statistical analysis of correlative clinical studies.
